(19) United States
(12) Patent Application Publication
(10) Pub. No.: US 2026/0174542 A1
(43) Pub. Date: Jun. 25, 2026

(54) IMPLANTABLE ARTIFICIAL LIGAMENT, AND PROCESS FOR MANUFACTURING SUCH AN ARTIFICIAL LIGAMENT

(71) Applicant: COUSIN BIOTECH, WERVICQ SUD (FR)

(72) Inventors: Lucie BECQUART, Lille (FR); François AUBERT, TOURCOING (FR); Stéphane NOEL, HANTAY (FR); Michel CAILLIBOTTE, Lille (FR)

(21) Appl. No.: 19/127,541

(22) PCT Filed: Nov. 9, 2023
(86) PCT No.: PCT/EP2023/081265

§ 371 Date: May 6, 2025

(30) Foreign Application Priority Data
Nov. 9, 2022 (FR) FR2211649

(51) Int. Cl. A61F 2/08; A61B 17/70; A61F 2/44; A61F 2/46

(52) U.S. Cl. CPC A61F 2/08; A61B 17/7032; A61B 17/7059; A61F 2/0811; A61F 2/4455; A61F 2/4611

(57) ABSTRACT

The present invention relates to an artificial ligament (10) comprising a hollow textile sheath (20) receiving a deformable elastic insert offsetting the rigid deformation phase of said artificial ligament (10) when a longitudinal tensile force is applied thereto.

### TECHNICAL FIELD

The present invention relates to the technical field of implantable artificial ligaments, in particular for the prevention and/or treatment of a spinal pathology, more particularly for the prevention and/or treatment of pathologies occurring following fusion of one or more spinal vertebrae, preferably for the prevention and/or treatment of proximal junctional kyphosis (PJK).

The present invention also relates to the field of methods for manufacturing such artificial ligaments.

### PRIOR ART

After spinal surgery intended to fuse one or more vertebrae, the deformations of the spine during the patient's movements, particularly in flexion, are distributed over the remaining non-fused vertebrae, and therefore located close to the fused spinal zone. This arrangement concentrates the loads on the non-fused vertebrae and thus modifies the natural gradient of load distribution observed in a spine without a fused zone. By nature, the facet joints block posterior spinal migrations and the intervertebral discs block anterior spinal migrations. Moreover, these elements of the spine comprise joint capsules containing synovial fluid, which acts as a lubricant for the facets in order to prevent bone wear of the joints. After one or more vertebrae have fused, the facet joints near the fused zone no longer function properly. The loads are therefore transferred to the ligaments, which are not naturally configured to accept such a high cumulative load. The imbalances created in the spine can lead to kyphosis, or even spondylolisthesis in the most serious cases.

Proximal junctional kyphosis (PJK) can lead to a proximal junctional failure (PJF) which is a serious early complication of adult spinal surgery and may require revision surgery. Risk factors for spinal surgery include overcorrection, under-correction, excessive ligament dissection and osteoporosis. These pathologies (PJK, PJF) of the spine generally occur within three months of spinal surgery in around 66% of cases. Up to 40% of adult patients who have undergone spinal surgery suffer from PJK. Deterioration of PJK into PJF must be avoided, since PJF requires revision surgery, which represents discomfort and additional risks for the patient, and incurs additional costs.

Following one or more vertebral fusions, it is thus sought to recreate a natural load distribution gradient and to relieve the non-fused vertebrae, in particular to avoid anterior collapse of the fused zone.

It is known to use braided textile ligaments to eliminate the abrupt transition between thoracic rigidity after fusion of one or more vertebrae, particularly lumbar vertebrae, and the flexibility of non-fused vertebral segments. However, the proposed textile ligaments have a very rigid and insufficiently elastic behaviour. These ligaments do not adapt correctly to the transition between the rigidified fused zone and the adjacent non-fused zones.

There is therefore a need for an artificial ligament that behaves rigidly over certain deformation ranges and elastically over other deformation ranges in order to compensate for the thoracic rigidity compared with the flexibility of the non-fused vertebral segments.

### DISCLOSURE OF THE INVENTION

An object of the present invention is, according to a first aspect, an artificial ligament, in particular for the treatment of proximal junctional kyphosis (PJK), alleviating all or some of the aforementioned problems in that it comprises, advantageously substantially consists of, even more advantageously consists of, an elongate braided hollow sheath having an internal volume, and a deformable insert housed in the internal volume of the braided hollow sheath over at least part of the length of said hollow sheath.

Advantageously, the artificial ligament has:
a deformation phase A in which the leading coefficient ka measured over a curve Cₗₐ of tensile force (N) vs. elongation (%) of said artificial ligament is less than or equal to 6 Newtons/%, advantageously less than of equal to 5 Newtons/%; and
a deformation phase B in which the leading coefficient kb measured over said curve Cₗₐ of tensile force (N) vs. elongation (%) of said artificial ligament is greater than or equal to 4 Newtons/%, advantageously greater than or equal to 5 Newtons/% and less than or equal to 40 Newtons/%, the elongation of said artificial ligament in said deformation phase B is greater than or equal to 2% and less than or equal to 15%, in particular less than or equal to 10%;
a deformation phase C in which the leading coefficient kc measured over said curve Cₗₐ of tensile force (N) vs. elongation (%) of said artificial ligament is greater than the leading coefficient kb of phase B.

Advantageously, the provision of a deformable insert over all or part of the length of the internal volume of the hollow sheath makes it possible to modify the mechanical behaviour of said sheath. In particular, this deformable and elastic insert makes it possible to increase the capacity of the sheath, and therefore ultimately of the ligament, to elongate it and to create a range of rigidity, particularly suitable for treating PJK.

Advantageously, in phase A, corresponding to low deformations, in particular an elongation (%) less than or equal to 5% or 2%, a low elongation is observed under a low load of the artificial ligament, then an elongation which increases slightly for a high load.

A non-exhaustive and non-limiting technical explanation of the present invention is that in phase A, the behaviour of the artificial ligament is dominated by and therefore largely attributable to the deformable insert. Then, in phase B, the yarns of the hollow textile sheath continue to slide relative to each other, compressing the deformable insert and increasing the force applied and the elongation obtained. Finally, in the last phase, i.e. phase C, the deformable insert is completely compressed/crushed by the yarns of the hollow sheath, and the behaviour of the artificial ligament is imparted by said yarns of the hollow sheath. The behaviour in phase C corresponds to the so-called rigid behaviour phase of the artificial ligament.

The deformable insert thus enables the rigid behaviour of the hollow sheath to be offset to high elongations, while retaining flexible/elastic behaviour at low or even very low elongations. This arrangement makes it possible to impart flexibility to non-fused vertebrae located close to fused vertebrae for small amplitude movements, and rigidity for larger movements.

Advantageously, the artificial ligament is implantable, in particular it is suitable for being inserted into a living organism (human or animal) for a prolonged period, ranging for example from a few days to several months or years.

In an embodiment, the curve Cₗₐ of tensile force vs. elongation of said artificial ligament is measured on a Lloyd Lrx Plus machine, with a traction speed of 100 mm/minute, in particular at ambient temperature, for example at a temperature ranging from 19° C. to 23° C., in particular without any particular relative humidity condition, optionally a preload of 1 N to 10 N is applied, in particular at a preload speed of 50 mm/min. The artificial ligament comprises, in particular, first and second ends; a first end is disposed in first jaws, and a second end is disposed in second jaws, the first jaws are fixed while the second jaws move in translation at a speed of 100 mm/minute.

Preferably, the initial test length of the artificial ligament is 395 mm +/−5 mm.

Preferably, the load sensor is a 5000 N load sensor.

In particular, the Lloyd Lrx Plus machine is a single-column testing machine, more specifically available in the Plus/Easy Test series of the Lloyd Instruments Materials testing range marketed by AMETEK. It preferably has a standard RS232 computer interface. Preferably, the force transducer has reference XLC-5000-A1, with an accuracy of 0.5% up to 5000 N, in particular model 01/2364.

In one embodiment, the deformable insert is elastic.

The tensile force applied to said insert or ligament or to said braided hollow sheath is always considered in the present text to be applied in the longitudinal direction of said insert or ligament or braided hollow sheath.

Preferably, the values of elongation (%) and tensile force measured and indicated in the present text on the curve Cₗₐ for tensile force (N) vs. elongation (%) of said artificial ligament are obtained during the first traction cycle, i.e. after the first traction, carried out on the artificial ligament after it has been removed from its sterilised packaging.

Preferably, the tensile forces and elongations (%) given herein are not tensile forces causing break, and therefore elongations at break, of the ligament, unless otherwise specified.

In this text, “elastic insert” means an insert having a residual deformation of less than or equal to 10% for an applied longitudinal force of less than or equal to 300 N, preferably less than or equal to 200 N, and even more preferably less than or equal to 100 N. The elastic insert thus deforms longitudinally under a maximum applied force of 300 N, and recovers its initial length, undeformed or with a variation of only 10% at most, advantageously by 8% at most, even more advantageously by 5% at most, in particular by 3% at most. The deformation can be calculated as follows: (Residual length of the deformable insert after at least one longitudinal deformation-Initial length of the deformable insert having undergone no longitudinal deformation)/Initial length of the deformable insert having undergone no longitudinal deformation.

### Artificial Ligament

Preferably, the artificial ligament comprises first and second ends, in particular free ends, even more preferably between which the longitudinal body of said artificial ligament extends. Preferably, the artificial ligament comprises a length L, and a width I (if it is flat) or a diameter d (if it is substantially round).

Preferably, the length L of the artificial ligament is greater than or equal to 50 mm and less than or equal to 400 mm, even more preferably greater than or equal to 100 mm and less than or equal to 300 mm.

Preferably, the width I or diameter d of the artificial ligament is greater than or equal to 3 mm and less than or equal to 20 mm, even more preferably less than or equal to 15 mm, preferably less than or equal to 10 mm.

Preferably, the artificial ligament has a longitudinal axis Lₗₐ, advantageously extending between its first and second ends, in particular free ends.

Preferably, the artificial ligament has a transverse axis Tₗₐ substantially secant to the axis Lₗₐ, in particular substantially perpendicular to the axis Lₗₐ.

The artificial ligament may be elongate and flat or elongate and substantially round.

Preferably, the elongation at break of the artificial ligament is less than or equal to 50%, in particular less than or equal to 40%, more particularly greater than or equal to 10% or 20%. Even more preferably, the braided hollow sheath comprises polyethylene terephthalate yarns.

### Hollow Sheath

Preferably, the hollow sheath comprises first and second ends, even more preferably between which the longitudinal body of the hollow sheath extends.

Preferably, the hollow sheath has a length L_{gc} and a width l_{gc} (if it is flat) or a diameter d_{gc} (if it is substantially round).

Preferably, the length L_{gc} of the hollow sheath is greater than or equal to 50 mm and less than or equal to 400 mm, even more preferably greater than or equal to 100 mm and less than or equal to 300 mm.

Preferably, the width l_{gc} or the diameter dgc of the hollow sheath is greater than or equal to 3 mm and less than or equal to 20 mm, even more preferably less than or equal to 15 mm, preferably less than or equal to 10 mm.

Preferably, the hollow sheath has a longitudinal axis L_{gc} and transverse axis T_{gc}, in particular substantially perpendicular to the axis L_{gc}.

The hollow sheath may be elongate and flat or elongate and substantially round.

The hollow sheath is braided and comprises a plurality of braided yarns.

Preferably, the hollow sheath comprises, advantageously substantially consists of, even more advantageously consists of, one or more monofilament yarns, and/or spun fibre yarns, and/or multifilament yarns, preferably one or more multifilament yarns.

The hollow sheath has an internal volume, in particular delimited by its inner wall, substantially opposite its outer wall. Said internal volume accommodates said insert over all or part of its length L_{gc}.

The hollow sheath comprises, advantageously substantially consists of, even more advantageously consists of, at least one yarn or a plurality of yarns which is/are totally or only partially bioresorbable or non-resorbable, preferably non-resorbable.

Preferably, the braided hollow sheath comprises, advantageously substantially consists of, even more advantageously consists of, several braided strands, in particular a braided roving is supported by a spindle on the braiding machine, each braided roving comprising one or more yarns, in particular as described in this text.

Preferably, the braided hollow sheath comprises, advantageously substantially consists of, even more advantageously consists of, a number of braided strands ranging from 8 to 64, even more preferably ranging from 12 to 52 strands, preferably comprising from 16 to 48 strands.

Preferably, the hollow sheath, in particular braided hollow sheath, comprises, advantageously substantially consists of, even more advantageously consists of, one or more yarns comprising at least one material selected from the list comprising: polyethylene terephthalate, high-tenacity polyethylene terephthalate, polybutylene terephthalate, high-molecular-weight polyethylene, ultra-high-molecular-weight polyethylene, polyamide 4-6 or 6-6, . . . or one of the mixtures thereof, preferably from the list comprising: polyethylene terephthalate, in particular high-tenacity polyethylene terephthalate, high-or ultra-high molecular weight polyethylene (e.g. HMWPE or UHMWPE).

In the present text, bioresorbable means the capacity of a material (for example a textile, a yarn, a foam, or a coating, etc.) to be degraded by a living organism in which it is implanted so that it disappears at the end of a given period, for example after 10 days or after several months, for example after 6 months.

### Deformable Insert

Preferably, the insert comprises first and second ends, even more preferably between which the longitudinal body of the insert extends.

The insert can be continuous or discontinuous. When the insert is discontinuous, the insert comprises a plurality of insert sections arranged adjacently or spaced apart in the internal volume of the hollow sheath.

Preferably, the insert has a length Li and a width li (if it is flat) or a diameter di (if it is substantially round).

Preferably, the length Li of the insert is greater than or equal to 50 mm and less than or equal to 400 mm, even more preferably greater than or equal to 100 mm and less than or equal to 300 mm.

Preferably, the width li or diameter di of the hollow sheath is greater than or equal to 3 mm and less than or equal to 20 mm, even more preferably less than or equal to 15 mm, preferably less than or equal to 10 mm.

Preferably, Lᵢ is less than or equal to L_{gc}.

Preferably, Lᵢ or dᵢ is less than or equal to L_{gc} or dgc.

Preferably, the insert has a longitudinal axis Lᵢ and transverse axis Tᵢ, in particular substantially perpendicular to the axis Lᵢ.

The insert may be elongate and flat or elongate and substantially round.

The insert is preferably not a braid.

Preferably, the insert is non-fibrous (i.e. does not comprise yarns and/or fibres). Preferably, the insert comprises one or more elastomer materials.

In the present text, “elastomer material” means any material having an elongation at break greater than or equal to 200% or 300% or 400%, preferably greater than or equal to 500%, for example measured with standard ISO 37:2017 entitled Rubber, vulcanized or thermoplastic—Determination of tensile stress-strain properties.

Preferably, the insert comprises (in particular is) an elongate element comprising one or more yarns, and/or one or more foam polymer cords, and/or one or more rods, and/or one or more extruded polymer profiles, and/or one or more woven and/or knitted textile tapes/cords, and/or one or more non-woven tapes/cords.

Preferably, the insert comprises (in particular is) one or more polymer rods (in particular substantially cylindrical, in particular solid and/or hollow) and/or one or more extruded polymer profiles (in particular multi-lobed and/or star-shaped and/or spring-loaded), in particular comprising one or more elastomers.

A hollow rod is advantageously a tube.

The insert is preferably a rod or an assembly of several rods, in particular having (in particular each having) a substantially cylindrical cross-section. When the insert is an assembly of several rods, said rods are arranged parallel to one another.

Preferably said at least one rod, or each of said rods, and/or said at least one extruded polymer profile, or each of said extruded polymer profiles, comprises, advantageously substantially consists of, even more advantageously consists of, one or more materials selected from: polyurethane, polycarbonate-urethane, or polydimethylsiloxane, or a mixture thereof.

Preferably, said one or more materials selected from: polyurethane, polycarbonate-urethane, or polydimethylsiloxane, or a mixture thereof, is/are elastomers.

Preferably, said one or more elastomers have a Shore A hardness greater than or equal to 20 and less than or equal to 90, even more preferably greater than or equal to 50 and less than or equal to 80, in particular greater than or equal to 70 and less than or equal to 80.

The Shore A hardness can be measured using standard ISO 48-4:2018, entitled Rubber, vulcanized or thermoplastic—Determination of hardness—Part 4: Indentation hardness by durometer method (Shore hardness)

In one embodiment, the polycarbonate urethane (PCU) has a Shore A hardness greater than or equal to 80 and less than or equal to 90. In a preferred example, the polycarbonate urethane is selected from those marketed by DSM under the brand name Bionate®.

In one embodiment, the polydimethylsiloxane is an elastomer, again preferably with a Shore A hardness greater than or equal to 20 and less than or equal to 80. In a preferred example, the polydimethylsiloxane is marketed by Nusil.

In one embodiment, the deformable insert does not occupy the entire internal volume of the hollow sheath.

In one embodiment, the hollow sheath has an internal volume Vint, and the solid volume occupied by the deformable insert represents at most 95%, preferably at most 90%, even more preferably at most 85%, more preferably at most 80%, in particular at most 75%, most particularly at most 70%, of the internal volume Vint of the hollow sheath.

The “solid volume” of the deformable insert is understood to be any volume occupied by the deformable insert that does not include a void, for example not including the orifices of a foam of the deformable insert (i.e. all the solid material).

Preferably, the volume Vint of the hollow sheath is calculated theoretically as a function of the dimensions (mm) of the hollow sheath (internal diameter or internal width and internal height, internal length).

In one embodiment, the hollow sheath has an internal volume Vint, and the solid volume occupied by the deformable insert represents at least 25%, preferably at least 35%, even more preferably at least 45%, more preferably at least 55%, in particular at least 65%, more particularly at least 75%, of the internal volume Vint of the hollow sheath.

Advantageously, since the internal volume is not filled by the deformable insert, voids remain in the internal volume which will be compressed during longitudinal deformation of the ligament, thus delaying the engagement of the hollow sheath in the longitudinal deformation and allowing the insert to be stressed first.

The deformable insert may be totally or only partially bioresorbable or non-resorbable, preferably non-resorbable.

In a first embodiment, the deformable insert and/or the braided sheath (each) comprise (advantageously each substantially consists of, even more advantageously consists of) one or more yarns, preferably multifilament yarns, made of high or ultra-high molecular weight polyethylene (in particular ultra-high molecular weight or UHMWPE), and/or having at least one of the following properties:
a linear density (dtex) greater than or equal to 200 dtex, preferably greater than or equal to 200 dtex and less than or equal to 600 dtex, in particular greater than or equal to 300 dtex and less than or equal to 550 dtex, more particularly greater than or equal to 400 dtex and less than or equal to 500 dtex; and/or
a tenacity (cN/dtex) greater than or equal to 25 cN/dtex, preferably greater than or equal to 25 cN/dtex and less than or equal to 50 cN/dtex, in particular greater than or equal to 30 cN/dtex and less than or equal to 45 cN/dtex, more particularly greater than or equal to 30 cN/dtex and less than or equal to 40 cN/dtex; and/or
an elongation at break of less than or equal to 30%, preferably less than or equal to 20%, even more preferably less than or equal to 10%, preferably less than or equal to 5%; and/or
a breaking force greater than or equal to 80 N, preferably greater than or equal to 100 N, even more preferably greater than or equal to 140 N, preferably greater than or equal to 160 N, in particular less than or equal to 1000 N.

In a second embodiment, optionally in combination with the first embodiment, the deformable insert and/or the braided sheath (each) comprise (advantageously each substantially consists of, even more advantageously each substantially consists of) one or more yarns, preferably multifilament yarns, made of polyethylene terephthalate, and/or having at least one of the following properties:
a linear density (dtex) greater than or equal to 25 dtex, preferably greater than or equal to 25 dtex and less than or equal to 150 dtex, in particular greater than or equal to 25 dtex and less than or equal to 100 dtex, more particularly greater than or equal to 25 dtex and less than or equal to 65 dtex; and/or
a tenacity (cN/dtex) greater than or equal to 2.5 cN/dtex, preferably greater than or equal to 2.5 cN/dtex and less than or equal to 10.0 cN/dtex, in particular greater than or equal to 4 cN/dtex and less than or equal to 9.0 cN/dtex, more particularly greater than or equal to 6 cN/dtex and less than or equal to 9.0 cN/dtex; and/or
an elongation at break of less than or equal to 50%, preferably less than or equal to 40%, even more preferably less than or equal to 30%, preferably less than or equal to 20%; and/or
a breaking force greater than or equal to 150 cN, preferably greater than or equal to 250 cN, even more preferably greater than or equal to 300 cN, in particular less than or equal to 1000 CN.

Preferably, the linear density is measured using standard EN 13392, in particular dating from 2001 in the case of a monofilament yarn, or according to standard EN ISO 2060 dating from 1995 in the case of a multifilament yarn.

Preferably, the elongation at break, the tenacity and the breaking force are measured using standard EN 13895, in particular dating from 2003, in the case of a monofilament yarn, or according to standard EN ISO 2062 dating from 1993 in the case of a multifilament yarn.

In one embodiment, the braided hollow sheath comprises one or more polyethylene terephthalate yarns, in particular of high tenacity, the mass fraction of which in said sheath is greater than 0% and less than or equal to 50% or 40% or 30% or 20% or 10%.

In one embodiment, the braided hollow sheath comprises one or more polyethylene yarns, in particular of high or ultra-high molecular weight, the mass fraction of which in said hollow sheath is greater than or equal to 50% or 60% or 70% or 80% or 90%.

In one embodiment, the braided hollow sheath comprises one or more polyethylene yarns, in particular of high or ultra-high molecular weight, and one or more polyethylene terephthalate yarns, in particular of high tenacity, the mass fraction (FPE) of polyethylene yarns is greater than the mass fraction (FPET) of polyethylene terephthalate yarns in the hollow sheath, in particular the ratio between the mass fractions FPE: FPet varies from 90:10 to 60:40.

Advantageously, polyethylene is more resistant to abrasion than polyethylene terephthalate because the coefficient of friction of polyethylene is lower than that of polyethylene terephthalate. The presence of a large quantity of polyethylene makes it possible to improve the longevity of the artificial ligament, which undergoes wear when it is tensioned on the arthrodesis rods attached to the vertebrae.

Preferably, the insert is elongate, and even more preferably the length of the insert is substantially equal to the length of the braided hollow sheath.

Preferably, the insert and the braided hollow sheath have coincident central longitudinal axes Lin and L_{gc} respectively.

Preferably, the deformable insert and the braided hollow sheath are coaxial.

Preferably, the deformation phases take place in the order A, then B and finally C, the forces and elongations increasing from phase A to phase B and then phase C.

Preferably, phases A, B and C are distinct from each other.

Preferably, the artificial ligament exhibits deformation phases A, then B and finally C under the application of an increasing force (N).

Preferably, the curve Cₗₐ is the curve LAI1, or LAI2, or LAI3, or LAI4 or LAI5, in particular shown in FIGS. 8 and 9.

The term “leading coefficient (ka)” is understood to mean the leading coefficient of a tangent line TA at any point on curve C in phase A, preferably at any point on curve Cₗₐ for which the elongation is less than or equal to 7% or 5% or 2%.

The term “leading coefficient (kb)” is understood to mean the leading coefficient of a tangent line TB at any point on curve Cₗₐ in phase B, preferably at any point on curve Cₗₐ for which the elongation is greater than or equal to 5% or 2%, even more preferably less than or equal to 15%, preferably less than or equal to 10% or 9% or 8% or 7% or 4%.

The term “leading coefficient (kc)” is understood to mean the leading coefficient of a tangent line TC at any point on curve C in phase C, preferably at any point on curve C for which the elongation is greater than or equal to 4% or 5%, preferably greater than or equal to 6% or 7%, even more preferably greater than or equal to 10%, and optionally greater than or equal to 15%.

Preferably, the leading coefficient (kc) is the leading coefficient of a tangent line TC at any point on curve C in phase C, at any point on curve C for which the elongation is less than or equal to 30% or 20%.

The expression “substantially consists of” is understood to mean that the one or more elements introduced by this expression is/are present in the majority, optionally at 95% by mass; it is possible that one or more other (undescribed) elements are present but they are not in the majority.

In one embodiment, the deformable insert is a rod comprising a thermoplastic or thermosetting elastomer, preferably comprising (advantageously substantially consisting of, even more advantageously consisting of) one or more polymers selected from: polyurethane, polycarbonate-urethane, polydimethylsiloxane, or a mixture thereof.

In the present text, “thermoplastic elastomer” is understood to mean that said elastomer present in the insert can be transformed by heating (in particular moulded, extruded or injected), in particular said elastomer has a softening or melting temperature allowing it to be hot-formed.

In the present text, “thermosetting elastomer” is understood to mean that said elastomer present in the insert cannot be transformed by heating, in particular said elastomer has no melting temperature.

In one embodiment, the insert is a rod or an assembly of rods, and each rod substantially consists of, in particular consists of, one or more polymers selected from the following polymers, in particular the following elastomers: polyurethane, polycarbonate-urethane, polydimethylsiloxane or a mixture thereof.

In particular, said polymer is a polyurethane or polycarbonate-urethane or polydimethylsiloxane.

In one embodiment, said insert is a rod or an assembly of rods, in particular said rod or each of said rods is extruded.

In one variant, the largest dimension (d) of the cross-section of said rod or each of said rods, or the diameter (d) when the cross-section is substantially circular, is d, which is greater than or equal to 1 mm and less than or equal to 5 mm.

In an alternative embodiment, said artificial ligament has an elongation less than or equal to 15%, optionally less than or equal to 10%, under the affect of a tensile force of order 250 Newtons or at 200 Newtons, preferably after at least 10 traction cycles, each traction cycle comprising the application of a tensile force of order 250 Newtons or 200 Newtons.

In an alternative embodiment, the difference (%) between the residual elongation (%) obtained after a traction cycle (n) of the ligament and the residual elongation (%) obtained after a traction cycle (n+1) of the ligament, with n is a integer different from 0, preferably n is equal to 10, is less than or equal to 15%, preferably less than or equal to 10%, more preferably less than or equal to 8%, preferably less than or equal to 5%, even more preferably less than or equal to 3%, each traction cycle comprising the application of a tensile force of order 250 Newtons or 200 Newtons.

The tensile force of order 250 Newtons or 200 Newtons is less than the ultimate tensile strength of the artificial ligament.

The integer n corresponds to the traction cycle number.

A traction cycle in the present text comprises the application of the tensile force, here of order 250 N or 200 N, then return to the rest state of the ligament, i.e. without exerting the tensile force on the ligament.

In the present text, the term “residual elongation (%)” is understood to mean the difference between the residual length (mm) of the artificial ligament after one traction cycle (n) and the initial length (mm) of the artificial ligament (advantageously before the application of any traction cycle) divided by the initial length (mm) of the artificial ligament ((residual length of the ligament-initial length of the ligament)/initial length of the ligament).

In an alternative embodiment, in deformation phase A, the elongation of said artificial ligament is less than or equal to 7% or 5%, optionally less than or equal to 2%, under the application of a tensile force less than or equal to 200 Newtons; and in deformation phase C, the elongation of said artificial ligament is greater than or equal to 5%, preferably greater than or equal to 10%, more preferably greater than or equal to 15%, preferably less than or equal to 30%, under the application of a tensile force greater than or equal to 200 Newtons and less than or equal to 800 Newtons.

In an alternative embodiment, in particular in deformation phase A, the elongation of said artificial ligament is less than or equal to 5%, optionally less than or equal to 2%, under the application of a tensile force less than or equal to 150 Newtons, preferably less than or equal to 100 Newtons, more preferably less than or equal to 50 Newtons, preferably less than or equal to 40 Newtons or 30 Newton or 20 Newtons or 15 Newtons or 10 Newtons.

Advantageously, in deformation phase A, the elongation of said artificial ligament is less than or equal to 5%, optionally to 2%, under the application of a tensile force greater than 0 Newtons, advantageously greater than or equal to 5 newtons or 10 Newtons.

In an alternative embodiment, in particular in deformation phase C, the elongation of said artificial ligament is greater than or equal to 5%, advantageously greater than or equal to 10%, even more advantageously greater than or equal to 15%, in particular less than or equal to 50% or 40%, under the application of a tensile force greater than or equal to 200 or 300 Newtons, in particular less than or equal to 300 Newtons or 250 Newtons.

In an alternative embodiment, in particular in phase A, the elongation of the artificial ligament is less than or equal to 6% or 5% or 4% or 2%, under the application of a tensile force less than or equal to 50 Newtons, preferably less than or equal to 40 or 30 or 20 or 15 or 10 Newtons (in particular greater than 0 N).

In an alternative embodiment, in particular in phase B, the elongation of the artificial ligament is greater than or equal to 2% or 5% or 7%, preferably less than or equal to 10% or 9% or 8% or 7% or 4%, under the application of a tensile force greater than or equal to 10 or 20 or 30 or 40 or 50 Newtons, preferably less than or equal to 100 or 70 or 60 or 50 Newtons.

In an alternative embodiment, in particular in phase C, the elongation of the artificial ligament is greater than or equal to 4% or 5% or 6% or 7% or 8%, preferably less than or equal to 15% or 10%, under the application of a tensile force greater than or equal to 100 N or 150 N or 200 N or 250 N, preferably less than or equal to 400 N or 350 N or 300 N or 250 N or 200 N or 150 N.

In an alternative embodiment, kb is greater than ka, in particular kb is greater than or equal to twice ka, more particularly kb is greater than or equal to three times ka.

The foot of the curve is very important when moving from phase A to phase B, because the force applied in phase A for very low elongations (of order a few %) is close to 0 Newtons.

In an alternative embodiment, kc is greater than kb, in particular kc is greater than or equal to twice kb, more particularly kc is greater than or equal to three times kb, even more particularly kc is greater than or equal to four times kb.

There is an increase in rigidity in phase C of the deformation of the artificial ligament, in which the mechanical properties of the sheath yarns are mainly expressed. The foot of the curve is very significant during the transition from phase B to phase C.

In an alternative embodiment, in the deformation phase B, the tensile force applied to the artificial ligament is less than or equal to 200 Newtons, preferably less than or equal to 100 Newtons.

In an alternative embodiment, the internal volume of said at least one part of the braided hollow sheath receiving said insert comprises one or more empty cavities which can be compressed so as to collapse when passing from deformation phase A to deformation phase B.

Advantageously, the empty cavities are reversibly compressible so that when passing from phase B to phase A, and optionally at rest, said cavities recover their initial volumes.

Said one or more cavities may each have a dimension in the micrometre or millimetre range. In an alternative embodiment, the insert has a void cavity fraction greater than or equal to 30%, preferably less than or equal to 90%, even more preferably less than or equal to 80%, preferably less than or equal to 70%.

The void fraction of the insert is preferably calculated using the following formula: 100×[1(P/(M×S))] in which P is the linear mass of the insert in gram/cm, M is the density of the material forming the insert in gram/cm³ (for example of the polymer material in which the insert is formed), and S is the area of the cross-section of the insert in cm².

In an alternative embodiment, the solid surface area of the cross-section of the insert is less than the total surface area (mm²) of the cross-section of said at least one part of the braided hollow sheath receiving said insert in its internal volume.

Advantageously, the artificial ligament comprises empty spaces that can be crushed during its gradual elongation under the application of a longitudinal tensile force.

In an alternative embodiment, the insert is selected from: a tube, a foam cord, a deformable profile, and at least one elongate multi-lobed element, in particular a three-lobed or four-lobed element, for example at least one multi-lobed monofilament Advantageously, the insert has an irregular structure capable of creating cavities and/or comprises a foamed material comprising cavities.

Preferably, the deformable profile is a spring-loaded profile.

In an alternative, the insert is selected from: a tube, a foam, and at least one elongate element, in particular at least one rod or at least one monofilament yarn, more particularly a multilobe yarn.

In an alternative, the insert is a solid or hollow cylindrical polymer rod or a multi-lobed or spring-loaded extruded polymer profile, in particular a three-lobed or four-lobed profile.

Preferably, the tube has an inner diameter greater than or equal to 0.50 mm, or 0.75 mm, and less than or equal to 3 mm, preferably less than or equal to 2.5 mm or 2 mm or 1.5 mm.

Preferably, the tube has an outer diameter greater than or equal to 0.50 mm, or 0.75 mm, and less than or equal to 3 mm, preferably less than or equal to 2.50 mm or 2 mm or 1.5 mm. Preferably, the wall thickness of the tube is greater than or equal to 0.10 mm, preferably greater than or equal to 0.20 mm, even more preferably less than or equal to 1 mm, preferably less than or equal to 0.50 mm or 0.40 mm or 0.30 mm, for example approximately 0.25 mm to within +/−0.05 mm.

Preferably, the cylindrical or multi-lobed rod has a diameter greater than or equal to 1 mm, and less than or equal to 5 mm or 4 mm or 3 mm, for example approximately 2 mm to within +/−0.5 mm. In one exemplary embodiment, the insert is a hollow cylindrical rod, i.e. a tube, comprising polyurethane having a Shore D hardness ranging from 50 to 75, in particular 65 Shore D to within +/−5, for example the brand Tecothane® marketed by Lubrizol.

In an alternative embodiment, the deformable insert has an elongation greater than or equal to 200% or 300%, and less than or equal to 500% or 400%, in particular for a tensile force greater than or equal to 10 N and less than or equal to 100 N, in particular less than or equal to 60 N or 50 N, or for a deformation stress greater than or equal to 10 MPa and less than or equal to 150 MPa or 100 MPa.

In an alternative embodiment, the insert is an elongate element that is extruded, foamed or 3D printed, in particular by additive or subtractive manufacturing or a combination thereof.

In an alternative embodiment, said insert comprises at least one polymer material having a Shore A hardness ranging from 5 to 90, in particular ranging from 5 to 50 or from 50 to 80, more particularly ranging from 20 to 50.

Preferably, the Shore A hardness is determined in accordance with standard NF EN ISO 868 dated 2003.

Preferably, said insert has a Shore A hardness ranging from 5 to 90, in particular from 5 to 50, more particularly from 20 to 50.

Preferably, at least 50% by mass of said insert substantially consists of said at least one polymer material having a Shore A hardness ranging from 5 to 90, even more preferably at least 80% or 90% by mass of said insert substantially consists of said at least one polymer material having a Shore A hardness ranging from 5 to 90.

In an alternative embodiment, the braided hollow sheath comprises, advantageously substantially consists of, even more advantageously consists of, at least 10, preferably at least 14, braided strands, each of the strands comprising at least one multifilament yarn.

In an alternative embodiment, the braided hollow sheath comprises, advantageously substantially consists of, even more advantageously consists of, one or more yarns comprising one or more materials selected from: high or ultra-high molecular weight polyethylene, polyethylene terephthalate, polypropylene, polyether ether ketone (PEEK), polyether ketone ketone (PEKK), polytrimethylene terephthalate (PTT), or a combination thereof, preferably high or ultra-high molecular weight polyethylene, and polyethylene terephthalate, or a combination thereof.

In an alternative embodiment, the height of a braided helix pitch of the braided hollow sheath is greater than or equal to 4 mm, preferably greater than or equal to 8 mm, in particular between 4 mm and 25 mm.

A braided helix pitch corresponds to the distance separating two crossings of a given braided yarn with the longitudinal axis of the hollow braided sheath.

In an alternative embodiment, the outer diameter of the insert is greater than or equal to 0.50 mm, or 0.75 mm or 1 mm, or 2 mm, and less than or equal to 15 mm, preferably less than or equal to 10 mm, even more preferably less than or equal to 8 mm or 5 mm or 4 mm or 3 mm. An object of the present invention according to a second aspect is a method for manufacturing an artificial ligament, in particular according to any one of the alternatives/embodiments with reference to the first aspect of the invention, advantageously comprising:
a) providing a deformable insert,
b) a step of braiding a hollow sheath having an internal volume, at least part of which is overbraided around said deformable insert, said deformable insert being housed in the internal volume of the braided hollow sheath over at least part of the length of said hollow sheath, and the ligament has:
a deformation phase A in which the leading coefficient ka measured over a curve Cₗₐ of tensile force (N) vs. elongation (%) of said artificial ligament is less than or equal to 6 Newtons/%, preferably less than of equal to 5 Newtons/%; and
a deformation phase B in which the leading coefficient kb measured over said curve Cₗₐ Of tensile force (N) vs. elongation (%) of said artificial ligament is greater than or equal to 4 Newtons/%, preferably greater than or equal to 5 Newtons/% and less than or equal to 40 Newtons/%, the elongation of said artificial ligament in said deformation phase B is greater than or equal to 2% and less than or equal to 15%, in particular less than or equal to 10%;
a deformation phase C in which the leading coefficient kc measured over said curve Cₗₐ Of tensile force (N) vs. elongation (%) is greater than the leading coefficient kb of phase B.

In an alternative embodiment, said process comprises a heat-setting step comprising the application of a temperature ranging from 90° C. to 300° C., in particular of order 130° C., for at least 1 minute, in particular for a period ranging from 2 minutes to 10 minutes. Preferably, a load (applied to the free ends of said artificial ligament) ranging from 1 daN to 10 daN is applied to the artificial ligament during the heat-setting step.

In one embodiment, the braided hollow sheath comprises (in particular substantially consists of) one or more polyethylene yarns, and the temperature applied during the heat-setting step is greater than or equal to 90° C. and less than or equal to 300° C.

In one embodiment, the braided hollow sheath comprises (in particular substantially consists of) one or more polyethylene terephthalate or polybutylene terephthalate yarns, and the temperature applied during the heat-setting step is greater than or equal to 150° C. and less than or equal to 230° C.

### DESCRIPTION OF THE DRAWINGS

The present invention will be better understand on reading the following embodiments, cited by way of non-limiting examples, and illustrated by the figures, in which:

FIG. 1 schematically shows a first example of an artificial ligament according to the invention;

FIG. 2 schematically shows, in an enlarged view, the height of a braided helix pitch of the braided hollow sheath of the artificial ligament shown in FIG. 1;

FIG. 3 schematically shows a second example of an artificial ligament according to the invention;

FIG. 4 schematically shows a second example of a deformable insert suitable for implementing the invention;

FIG. 5 schematically shows a third example of a deformable insert suitable for implementing the invention;

FIG. 6 schematically shows a fourth example of a deformable insert suitable for implementing the invention;

FIG. 7 schematically shows a fifth example of a deformable insert suitable for implementing the invention;

FIG. 8 schematically shows various curves Cₗₐ in which the applied tensile force is indicated on the ordinate, and the elongation obtained (%) is indicated on the abscissa for flat artificial ligaments according to the invention, and a hollow braided sheath without insert forming a comparative artificial ligament; and

FIG. 9 schematically shows various curves Cₗₐ in which the tensile force applied is indicated on the ordinate, and the elongation obtained (%) is indicated on the abscissa for substantially tubular artificial ligaments according to the invention, and two hollow braided sheaths without an insert forming comparative artificial ligaments.

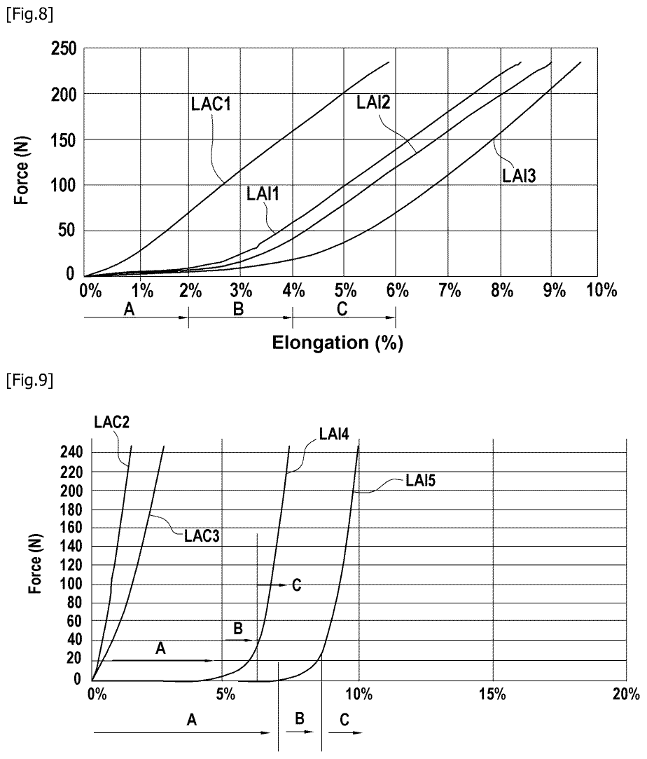

The curves shown in FIGS. 8 and 9 are obtained for traction cycles which do not go as far as ligament break, but for a maximum applied tensile force of approximately 250 Newtons. These curves Cₗₐ of tensile force vs. elongation of said artificial ligament are measured as described above in the present text, i.e. on a Lloyd Lrx Plus machine, with a traction speed of 100 mm/minute, in particular at ambient temperature, for example at a temperature ranging from 19° C. to 23° C., without any particular relative humidity condition, optionally a preload of 1 N to 10 N is applied, in particular at a preload speed of 50 mm/min. The artificial ligament comprises, in particular, first and second ends; a first end is disposed in first jaws, and a second end is disposed in second jaws, the first jaws are fixed while the second jaws move in translation at a speed of 100 mm/minute. Preferably, the initial test length of the artificial ligament is 395 mm +/−5 mm.

### DESCRIPTION OF THE EMBODIMENTS

The first example of an artificial ligament 10 according to the invention comprises a hollow braided sheath 20 defining an internal volume 22 and having a determined length Lgci and first and second opposite ends (24, 26). The artificial ligament 10 also comprises a deformable insert 30 having a length Lᵢ and first and second opposite ends (34, 36). In this particular example, the deformable insert 30 is a flat, solid polyurethane rod having a Shore A hardness of approximately 40. In particular, the length Lᵢ₁ is less than L_{gc1} but could be equal to L_{gc1}. The helix pitch Pₕ₁ of the hollow braided sheath 20 is of order 11 mm in this particular example. The braided helix pitch corresponds to the distance Pₕ₁ separating two adjacent crossings of a braided yarn—coloured black for identification purposes in FIG. 2—with the longitudinal axis of the hollow braided sheath 20.

In this specific example, the artificial ligament 10 comprises 16 strands, each comprising a multifilament polyethylene terephthalate yarn of 435 dtex and 120 filaments, said strands being braided on a 16-spindle braiding machine (one roving per spindle).

The second example of an artificial ligament 100 according to the invention comprises a hollow braided sheath 120 defining an internal volume 122 and having a determined length L_{gc2} and first and second opposite ends (124, 126). The artificial ligament 100 also comprises a deformable insert 130 having a length Lᵢ₂ and first and second opposite ends (134, 136). In this particular example, the deformable insert 130 is a solid cylindrical polyurethane rod having a Shore A hardness of approximately 40. In particular, the length Lᵢ₂ is less than L_{gc2} but could be equal to L_{gc2}.

The helix pitch Pₕ₂ of the hollow braided sheath 120 in this particular example is of order 19 mm.

In this particular example, the artificial ligament 100 comprises 48 strands each comprising 4 polyethylene terephthalate multifilament yarns, each of the yarns having a fineness of 138 dtex and comprising 32 filaments. The 48 strands are braided on a 48-spindle braiding machine (one roving per spindle).

In the present text, the number of braided strands corresponds to the number of spindles carrying one or more yarns (a roving may comprise one or more yarns) which are braided on the braiding machine.

The braided sheath 120 has more braided strands and is therefore less tight around the insert 130 than the braided sheath 20, which is very tight around the insert 30.

In order to offset the rigid behaviour of the hollow braided sheath under the effect of longitudinal tension, in particular in deformation phase C, the yarns of the sheath are braided so as to penetrate/engage the surface of the insert, i.e. with a helix pitch large enough for the yarns to slide relative to each other.

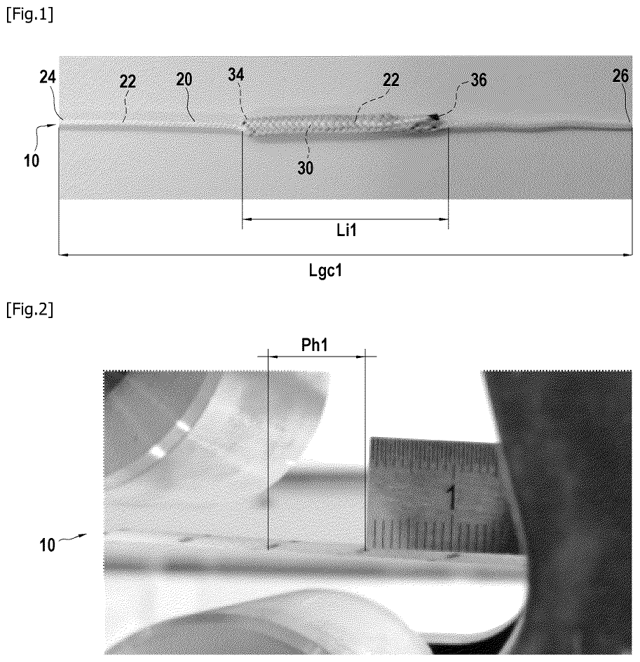

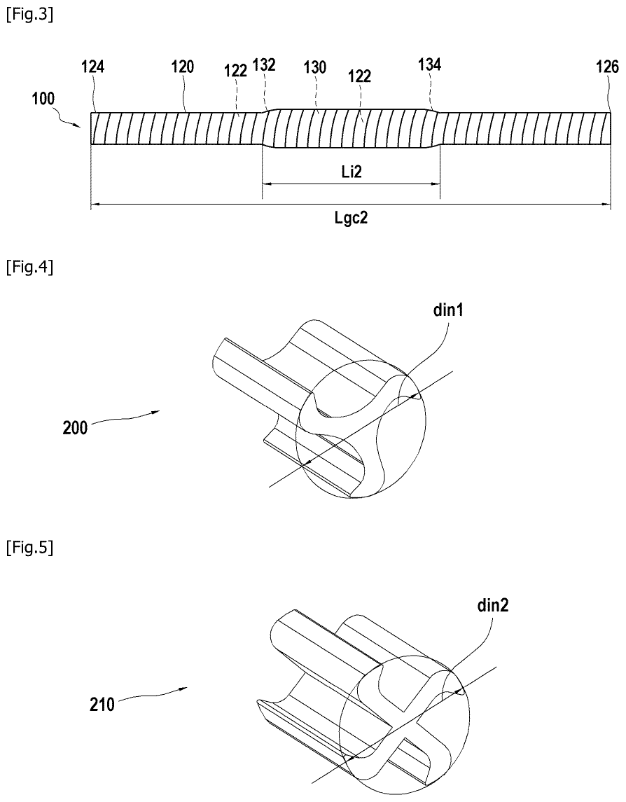

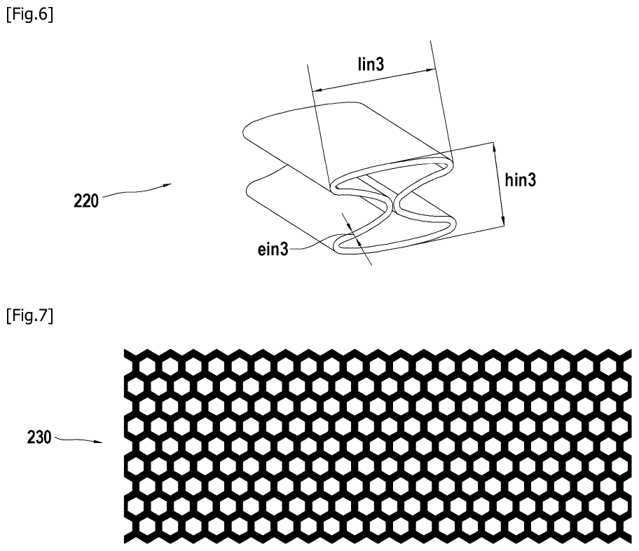

In the examples shown in FIGS. 1 to 3, the insert (30, 130) is a flat, solid rod made of elastic, deformable polyurethane. This insert can alternatively be, for example, an extruded or foamed polymer profile 200 having a three-lobed cross-section as shown in FIG. 4 or a four-lobed cross-section 210 as shown in FIG. 5, or a hollow rod such as a tube. The diameters dᵢₙ₁ and dᵢₙ₂ respectively of the inserts 200 and 210 (i.e. the diameter of the circle receiving the cross-section of said insert) are for example each of order 4 mm. The insert according to the invention may also be a spring-loaded profile 220 having a height hᵢₙ₃ ranging from 2 mm to 8 mm, for example of order 4 mm, a width lᵢₙ₃ ranging from 1 mm to 8 mm, for example of order 5 mm. The thickness of a wall eᵢₙ₃ ranges from 0.1 mm to 2 mm, for example is of order 0.4 mm. The insert according to the invention can also be a knitted honeycombed textile strip 230, wound on itself to form a tube around which the hollow sheath is braided.

Preferably, the insert according to the invention is a polyurethane or polycarbonate-urethane (PCU) or polydimethylsiloxane (PDMS) rod (in particular hollow or solid).

FIG. 8 shows the curves of tensile force applied longitudinally (N) and elongation obtained (%) for a comparative artificial ligament LAC1 and three artificial ligaments according to the invention whose hollow braided sheaths are substantially flat LAI1, LAI2 and LAI3, for a maximum applied tensile force of 250 Newtons.

### Example LAC1

This comparative artificial ligament comprises a hollow textile sheath braided on a 48-spindle braiding machine, and therefore comprises 48 textile strands, each textile roving comprises 4 polyethylene terephthalate yarns, each yarn comprises 32 filaments and has a titre of 138 dtex. This gives 552 dtex per roving. The helix pitch is approximately 19.64 mm.

### Example LAI1

This artificial ligament according to the invention comprises a hollow textile sheath braided on a 48-spindle braiding machine, and therefore comprises 48 textile strands, each textile roving comprises 4 polyethylene terephthalate yarns, each yarn comprises 32 filaments and has a titre of 138 dtex. This gives 552 dtex per roving. The helix pitch is approximately 19.64 mm. This artificial ligament comprises an insert formed from a flat, solid polyurethane rod with a Shore A hardness of approximately 40, having a length Lin of approximately 13.5 cm, a width lin of approximately 6.5 mm and a thickness of approximately 4 mm. The length of the hollow textile sheath L_{gc} is substantially equal to the length lin of the insert.

### Example LAI2

This artificial ligament according to the invention comprises a hollow textile sheath braided on a 48-spindle braiding machine, and therefore comprises 48 textile strands, each textile strand comprises 4 polyethylene terephthalate yarns, each yarn comprises 32 filaments and has a titre of 138 dtex. This gives 552 dtex per strand. The helix pitch is approximately 19.64 mm. This artificial ligament comprises an insert formed from a flat, solid polyurethane rod with a Shore A hardness of approximately 40, having a length Lin of approximately 10.5 cm, a width lin of approximately 5 mm and a thickness of approximately 4 mm. The length of the hollow textile sheath L_{gc} is substantially equal to the length lin of the insert.

### Example LAI3

This artificial ligament according to the invention comprises a hollow textile sheath braided on a 48-spindle braiding machine, and therefore comprises 48 textile strands, each textile strand comprises 4 polyethylene terephthalate yarns, each yarn comprises 32 filaments and has a titre of 138 dtex. This gives 552 dtex per strand. The helix pitch is approximately 19.64 mm. This artificial ligament comprises an insert formed from a flat, solid polyurethane rod with a Shore A hardness of approximately 40, having a length Lin of approximately 10.5 cm, a width lin of approximately 5 mm and a thickness of approximately 4 mm. The length of the hollow textile sheath L_{gc} is substantially equal to the length lin of the insert.

Ligaments LAC1, LAI1; LAI2 and LAI3 all undergo a calendering step during which they are calendered and heated, in particular to 110° C. for 45 seconds, in order to flatten them and fix this shape. Ligaments LAC1, LAI1 and LAI2 also undergo a heat-setting step, unlike ligament LAI3 which does not undergo a heat-setting step.

In these specific examples, and in a non-limiting manner, the heat-setting step comprises heating the artificial ligament tensioned under a load (preferably applied to the free ends of said artificial ligament) ranging from 1 daN to 10 daN, in particular 10 daN. The heating time is greater than or equal to 2 minutes and less than or equal to 10 minutes, and heating temperature is greater than or equal to 90° C. and less than or equal to 300° C. When the braided hollow sheath is made of polyethylene yarns, the heating and therefore heat-setting temperature is preferably greater than or equal to 80° C. and less than or equal to 120° C. When the braided hollow sheath consists essentially of one or more polyethylene terephthalate or polybutylene terephthalate yarns, the heating and therefore heat-setting temperature is greater than or equal to 150° C. and less than or equal to 230° C.

It can be seen that the ligament LAC1 has no A and B phases and begins the most rigid phase C almost immediately after the application of the tensile force (N). The ligaments according to the invention, LAI1, LAI2 and LAI3, have their deformation phase C offset by the A and B deformation phases. Phase A is shown as a deformation plateau in which the ligament elongates at almost zero tensile force with low elongation, in particular substantially less than or equal to 2%.

Deformation phase B, intermediate between phases A and C, shows an elongation which remains moderate, less than or equal to 4% for a still fairly modest applied force, in particular less than or equal to 100 N. Finally, in deformation phase C, the ligaments LAI1, LAI2 and LAI3 elongate much more but at much higher applied forces, for example greater than or equal to 100 N.

On curve LAI1, for example, a leading coefficient ka of order 4 N/%, a leading coefficient kb of order 5.5 N/%, and a leading coefficient kc at a first point (bottom of the curve) of 27.07 N/% and at a second point (middle of the curve) of 41.68 N/% are measured.

FIG. 9 shows the curves of longitudinally applied tensile force (N) and the elongations obtained (%) for two comparative artificial ligaments LAC2 and LAC3, and two artificial ligaments according to the invention LAI4, LAI5, for which the hollow braided sheaths are substantially round, and for a maximum applied tensile force of 240 Newtons.

### Example LAC2

This comparative artificial ligament comprises a hollow textile sheath braided on a 16-spindle braiding machine, and therefore comprises 16 textile strands, each textile strand comprises 1 ultra-high molecular weight polyethylene, for example of the brand Spectra, each yarn comprises 120 filaments and has a titre of 435 dtex. This ligament does not comprise a deformable insert. The helix pitch Ph is 11 mm.

### Example LAC3

This comparative artificial ligament is similar to the LAC2 ligament except that the pitch Ph of the helix is 5 mm.

### Example LAI4

This artificial ligament according to the invention comprises a hollow textile sheath braided on a 16-spindle braiding machine, and therefore comprises 16 textile strands, each textile strand comprises 1 ultra-high molecular weight polyethylene yarns, for example of the brand Spectra, each yarn comprises 120 filaments and has a titre of 435 dtex. The helix pitch ph is 11 mm. This artificial ligament comprises a laminate wound on itself with a length Lin of approximately 10 cm and a width lin of approximately 6 mm. The length of the hollow textile sheath L_{gc} is substantially equal to the length lin of the insert. The pre-tension applied before the force-elongation curve is established is 200 g.

### Example LAI5

This artificial ligament according to the invention comprises a hollow textile sheath braided on a 16-spindle braiding machine, and therefore comprises 16 textile strands, each textile strand comprises 1 ultra-high molecular weight polyethylene yarns, for example of the brand Spectra, each yarn comprises 120 filaments and has a titre of 435 dtex. The helix pitch ph is 11 mm. This artificial ligament comprises a laminate (an open-type knitwork, such as that shown in FIG. 7) wound on itself with a length Lin of approximately 10 cm and a width lin of approximately 6 mm. The length of the hollow textile sheath L_{gc} is substantially equal to the length lin of the insert. The pre-tension applied before the force-elongation curve is established is 300 g.

The curve LAI4, for example, has a leading coefficient ka of almost 0 N/%, a leading coefficient kb of order 12 N/% and a leading coefficient kc of order 133.

The behaviour of the artificial ligaments LAI4 and LAI5 is similar to that of ligaments LAI1-3: a deformation phase A deformation is observed similar to a plateau, then the beginning of the foot of the curve in phase B in which elongation remains moderate under a force that is likewise moderate, and a stiffness that increases sharply in deformation phase C. The leading coefficient kb is therefore greater than or equal to 3 times the leading coefficient ka, and similarly the leading coefficient kc is greater than or equal to 3 times the leading coefficient kb.

1-17. (canceled)

18. An artificial ligament for the treatment of proximal junctional kyphosis, wherein said artificial ligament comprises an elongate braided hollow sheath having an internal volume, and a deformable insert housed in the internal volume of the braided hollow sheath over at least part of the length of said hollow sheath, wherein the artificial ligament has:
a deformation phase A in which a leading coefficient ka measured over a curve Cₗₐ of tensile force (N) vs. elongation (%) of said artificial ligament is less than or equal to 6 Newtons/%; and
a deformation phase B in which a leading coefficient kb measured over said curve Cₗₐ of tensile force (N) vs. elongation (%) of said artificial ligament is greater than or equal to 4 Newtons/% and less than or equal to 40 Newtons/%, the elongation of said artificial ligament in said deformation phase B is greater than or equal to 2% and less than or equal to 15%;
a deformation phase C in which a leading coefficient kc measured over said curve Cₗₐ of tensile force (N) vs. elongation (%) of said artificial ligament is greater than the leading coefficient kb of phase B.

19. The artificial ligament according to claim 18, wherein the deformable insert is a rod or an extruded polymer profile comprising a thermoplastic or thermosetting elastomer.

20. The artificial ligament according to claim 19, wherein the deformable insert is a rod or an extruded polymer profile comprising one or more polymers selected from polyurethane, polycarbonate-urethane, a polydimethylsiloxane, or a mixture thereof.

21. The artificial ligament according to claim 18, wherein the difference (%) between the residual elongation (%) obtained after a traction cycle n of said ligament and the residual elongation (%) obtained after a traction cycle n+1 of said ligament, where n is an integer different from 0, is less than or equal to 15%, each traction cycle comprising the application of a tensile force of order 250 Newtons.

22. The artificial ligament according to claim 18, wherein:
in deformation phase A, the elongation of said artificial ligament is less than or equal to 5% for a tensile force greater than 0 Newtons and less than or equal to 20 Newtons; and
in deformation phase C, the elongation of said artificial ligament is greater than or equal to 5% for a tensile force greater than or equal to 200 newtons and less than or equal to 800 Newtons.

23. The artificial ligament according to claim 22, wherein in deformation phase C, the elongation of said artificial ligament is less than or equal to 50% for a tensile force greater than or equal to 200 newtons and less than or equal to 800 Newtons.

24. The artificial ligament according to claim 18, wherein the leading coefficient kb is greater than the leading coefficient ka.

25. The artificial ligament according to claim 18, wherein the leading coefficient kc is greater than or equal to 3 times the leading coefficient kb.

26. The artificial ligament according to claim 18, wherein the elongation of the artificial ligament is less than or equal to 5% under the application of a tensile force less than or equal to 40 Newtons.

27. The artificial ligament according to claim 18, wherein the internal volume of said at least one part of the braided hollow sheath receiving said insert comprises one or more empty cavities which can be compressed so as to collapse when passing from deformation phase A to deformation phase B.

28. The artificial ligament according to claim 18, wherein a solid surface area (mm₂) of the cross-section of the insert is less than a total surface area (mm₂) of the cross-section of said at least one part of the braided hollow sheath receiving said insert in the internal volume of the braided hollow sheath.

29. The artificial ligament according to claim 18, wherein the insert is selected from: a tube, a foam, and at least one elongate element.

30. The artificial ligament according to claim 29, wherein said at least one elongate element is selected from: at least one rod, at least one monofilament yarn, at least one multilobe yarn.

31. The artificial ligament according to claim 18, wherein the insert is an elongate element that is extruded, foamed or 3D printed.

32. The artificial ligament according to claim 18, wherein said insert comprises one or more polymer materials having a Shore A hardness ranging from 5 to 90.

33. The artificial ligament according to claim 18, wherein the braided hollow sheath comprises at least 10 strands that are braided, each of the strands comprising at least one multifilament yarn.

34. The artificial ligament according to claim 18, wherein the braided hollow sheath comprises one or more yarns comprising at least one material selected from: ultra-high molecular weight polyethylene, polyethylene terephthalate, polypropylene, polyether ether ketone (PEEK), polyether ketone ketone (PEKK) and polytrimethylene terephthalate (PTT).

35. The artificial ligament according to claim 18, wherein the height of a braided helix pitch of the braided hollow sheath is greater than or equal to 4 mm.

36. The artificial ligament according to claim 18, wherein the outer diameter of the insert is greater than or equal to 0.5 mm and less than or equal to 15 mm.

37. A method for manufacturing an artificial ligament wherein said method comprises:
a) providing a deformable insert,
b) a step of braiding a hollow sheath, having an internal volume, around said deformable insert, said deformable insert being housed in the internal volume of the braided hollow sheath over at least part of the length of said hollow sheath, and wherein the ligament has:
a deformation phase A in which a leading coefficient ka measured over a curve Cₗₐ of tensile force (N) vs. elongation (%) of said artificial ligament is less than or equal to 6 Newtons/%; and
a deformation phase B in which a leading coefficient kb measured over said curve Cₗₐ of tensile force (N) vs. elongation (%) of said artificial ligament is greater than or equal to 4 Newtons/% and less than or equal to 40 Newtons/%, the elongation of said artificial ligament in said deformation phase B is greater than or equal to 2% and less than or equal to 15%;
a deformation phase C in which a leading coefficient kc measured over said curve Cₗₐ of tensile force (N) vs. elongation (%) of said artificial ligament is greater than the leading coefficient kb of phase B.
